(19) United States
(12) Patent Application Publication
(10) Pub. No.: US 2008/0057298 A1
(43) Pub. Date: Mar. 6, 2008

(54) Low friction particulate coatings

(75) Inventor: Michael J. Finley, Saint Louis Park, MN (US)

Correspondence Address: KAGAN BINDER, PLLC, SUITE 200, MAPLE ISLAND BUILDING, 221 MAIN STREET NORTH, STILLWATER, MN, 55082, US

(73) Assignee: SurModics, Inc.

(21) Appl. No.: 11/512,022

(22) Filed: Aug. 29, 2006

(51) Int. Cl. B32B 5/16; B32B 15/04; A61L 33/00; B05D 1/12; G11B 5/64

(52) U.S. Cl. 428/327; 428/411.1; 428/336; 428/457; 427/180; 427/2.1

(57) ABSTRACT

The invention provides a low-friction halogenated nano- or microparticulate coating and method for forming the coating on articles, such as implantable medical articles. The halogenated nano- or microparticles, desirably fabricated from PTFE, are present on the surface of the coating and covalently coupled to a coupling component, which facilitates formation of the coating. The coatings are biocompatible and can be formed on a selected portion of a medical device in a straightforward process. In some aspects the nano- or microparticulate coatings are formed on a system for the insertion of a medical device, wherein the system includes a catheter.

### FIELD OF THE INVENTION

The invention relates to low-friction coatings formed using halogenated polymeric nano- or microparticles. The invention also relates to insertable medical article including these low-friction coatings, the preparation of these articles, and uses thereof.

### BACKGROUND OF THE INVENTION

Numerous systems include components that are moved in relation to one another. In many cases, these systems can function more efficiently by using materials that reduce frictional forces, thereby facilitating movement of the components in the system.

One type of frictional force is static friction force, which is the initial resistance to movement of two components in contact with one another. Movement of one component occurs when the static friction force is overcome by application of force to at least one of the components. If the static friction force is high, the application of force can cause a sudden, rapid relative movement of the two surfaces, resulting in an imprecise and undesired movement of one or more components of the system. In order to overcome problems that arise from static friction forces, low friction surfaces be formed on one or more of the components of the system.

Low friction surfaces can improve system function in various technologies. Examples of such technologies include small and large scale machinery; apparatus having telescoping functions, such as cameras; apparatus having piston/cylinder combinations, including those using syringes, hydraulic and pneumatic parts; engines; optical systems, including fiber optic cable; recreational equipment having moving parts, such as those that have bearings; electronics having moving parts and apparatus for the manufacture of electronics; cookware or food preparation machinery; drive train systems; mechanical hosing; and medical devices, in particular medical devices that are inserted into the body.

Medical systems having components with low friction surfaces may improve the process of implantation or insertion of a medical component into the body. For example, many procedures involving the insertion of a medical device into a portion of the body involve the movement of a medical device against another device, or the movement of one part of a device in contact with another part of a device. As with many movable parts, the reduction of frictional forces between devices or device parts can facilitate a medical process.

For example, self-expanding stents are typically deployed from the inner lumens of catheters to a target location within the body for the treatment of a medical condition. Generally, self-expanding stents are loaded into the catheter in a contracted state, causing pressure to be exerted on the inner walls of the catheter. This pressure hinders axial movement of the stent due to frictional forces. These frictional forces can cause the translation of movements of the stent within the catheter to be erratic and imprecise. Without reduction in frictional forces, stent movement and deployment may create trauma to the endothelium and may cause improper placement of the stent. The process of stent deployment is further complicated given that control of stent deployment via stent push wires is typically carried out at the proximal end of the catheter (user end), often through a tortuous pathway to the target site.

Some traditional stent deployment systems include a sheath or sleeve for constraining the stent in a contracted state. When the distal portion of the catheter is at the target location, the sheath or sleeve is retracted to expose the stent. After the sheath is removed, the stent is free to self-expand, or be expanded with a balloon. In a coronary stent deployment system that utilizes a retractable sheath, the interaction of the sheath and guide catheter upon retraction can be problematic. This issue is commonly dealt with by making the retractable sheath long enough so that it will be contained in the guide catheter at all times. The retraction of the sheath increases system profile, reduces flexibility, and creates excess friction upon sheath retraction.

It is generally known in the art of insertable medical devices that hydrophilic polymers can reduce the frictional forces on the surfaces of insertable medical devices. When coated on the surface of devices, hydrophilic polymers can become lubricious. However, hydrophilic coatings can swell considerably in the presence of water and increase the profile of the device (such at the thickness of the catheter wall, thereby decreasing the inner diameter of the catheter).

Rather than using a hydrophilic “wet” low friction coating, the medical device may be fabricated from a material such as polytetrafluoroethylene (PTFE) which can provide a low friction “dry” surface. PTFE is well-known for its chemical resistance, high temperature stability, resistance against ultra-violet radiation, low friction coefficient and low dielectric constant, among other properties. As a result, it has found numerous applications in harsh physico-chemical environments and other demanding conditions. The use of PTFE parts in medical devices, such as catheters, is also known.

For example, some catheter bodies are formed using a PTFE liner to define the delivery lumen (i.e., the inner diameter of the catheter). The surface of a PTFE liner has a very low coefficient of friction, and can facilitate movement of leads, guidewires, or stents moved within the lumen of the catheter. The reduction of frictional forces is particularly important when the article is moved within non-linear portions of the lumen. In these non-linear portions, contact stresses against the wall surface are greatest. While a PTFE liner can provide a low-friction surface, it may stiffen the catheter and make it difficult to be bent during an insertion process. Furthermore, the fabrication of catheter bodies with PTFE liners is labor intensive.

It can be very difficult to integrate PTFE into or on a prefabricated article since many thermoplastic articles are formed from polymers having different properties than PTFE. PTFE has a very high melting point and is insoluble in almost all solvents at temperatures up to about 300° C. PTFE processed at such a high temperature to provide a flowable or soluble composition would destroy most conventional thermoplastics. Given this, in order to provide an article, such as a medical article, conventional methods involve the preparation of a PTFE part, which then must be fitted into a portion of the article in order to provide the article with a low friction surface. This process, however, can be time consuming and expensive.

The preparation of useful coatings for the surfaces of medical devices is challenging. Twisting or contortion of the device during use in the body may result in cracking, or peeling of the coating. Furthermore, since hydrophilic coatings have the potential to swell to a certain extent in an aqueous environment, the components of the coating can potentially become dislodged and lost from the coating if not sufficiently stabilized. Given these factors, the coatings should adhere sufficiently to the device. Further, the dimensions and modulus of the device can be affected by coatings that are excessively thick.

Coatings are often prepared using organic solvents or low molecular weight monomeric compounds, which in some cases present toxicity concerns. While it is generally desirable to remove all solvent or unreacted low molecular weight monomeric materials, these components may remain in the coating in trace amounts. It is often necessary to properly handle these materials and remove them if they remain in the formed coating.

The methods and coatings of the present invention address these types of problems that are encountered in the preparation or use of low friction surfaces on medical devices. In addition the methods and coatings of the present invention are applicable to a variety of other technologies outside the field of medical devices.

### SUMMARY

The present invention is related to articles having low friction nano- or microparticulate coatings and methods for forming these coatings. The coating can be used in a wide range of technologies, including medical and non-medical technologies.

In some aspects, the coatings of the present invention are formed on medical articles that are inserted into a portion of the body. The coatings can be used in medical procedures, wherein the coating reduces the friction associated with the movement of one medical article in contact with another medical article. In other aspects, the coatings of the present invention can be formed on medical articles that are not inserted into the body, such as on syringe/plunger combinations.

The coatings of the present invention comprise an outer layer that includes halogenated polymeric nano- or microparticles. The coating also includes a coupling component. The halogenated polymeric nano- or microparticles are coupled to the coupling component via a reacted pair. The reacted pair includes a first reacted group pendent from the microparticle and a second reacted group pendent from the first compound. The coupling component facilitates the formation of a relatively durable and uniform outer layer of the halogenated polymeric nano- or microparticles. In some aspects of the invention, the halogenated polymeric nano- or microparticles includes a perfluorinated polymer such as PTFE.

In some specific aspects, the coupling component comprises a polymer that is soluble in a polar solvent. In other aspects, the halogenated polymeric nano- or microparticles have a size of about 1 μm or less. In more specific aspects, the halogenated polymeric nanoparticles have a size in the range of about 50 nm to about 500 nm, or about 200 nm to about 300 nm.

The low-friction coatings are compliant and conformal, and therefore are well suited for use on flexible articles. In some aspects the coating is formed on a flexible medical article such as medical catheters. Catheters are typically subject to considerable manipulation and flexion following insertion in the body. The coatings can be subject to a considerable amount of flexion without risk that the coatings will experience significant cracking or delamination. In addition, the nano- or microparticulate coatings are relatively smooth and durable.

The invention also provides a method for preparing a low friction coating. The method includes the steps of (a) forming a first coated layer on the surface of the article comprising a coupling component having a first reactive group; and (b) disposing a halogenated polymeric nano- or microparticle on the first coated layer to form second coated layer, wherein the halogenated polymeric microparticle comprises a second reactive group. In step (b), the second reactive group present on the surface of the microparticle react with the first reactive groups and couple the microparticle to the first coated layer.

Accordingly, the present invention provides an improved method for forming a low friction surface on a portion of a medical article. In particular, the inventive methods represent a distinct advancement in the preparation of low friction PTFE surfaces on medical articles. The low friction surfaces, such as PTFE surfaces, can be prepared without requiring the fabrication and integration of a PTFE insert into the medical article. The methods of the invention therefore significantly reduce the labor and expense associated with the fabrication of medical articles having PTFE surfaces.

Furthermore, the method does not require the application of high heat (e.g., above 300° F.) to melt the PTFE in order to form the coating. In this regard, the coating can be formed on a wide range of thermoplastics having melting points that are lower than that of PTFE.

The materials of the low friction coating can be readily prepared or commercially obtained. These compositions can also be coated on the surface of medical articles with great ease, for example, by dip-coating, brush-coating, or sponge coating, and do not require the use of elaborate coating equipment or methods.

The methods of the invention are also advantageous in that a low-friction surface can be formed at one or more desired location(s) on the article. The coupling component can disposed at a desired location on the surface of an article to form the first coated layer, followed by the application of halogenated nano- or microparticles to form a low friction surface at a desired location. Such precision can generally not be achieved using a PTFE insert.

The methods of the invention are also associated with improved biocompatibility and increased safety. Organic solvents or high temperature processing steps are not required to form the low friction coating. In many modes of practice, the coating of the invention can be formed using aqueous solutions.

The low-friction surface can be prepared without causing a substantial increase in the thickness of the coated article. In many aspects, the low-friction surface can be prepared by forming a nano- or microparticulate coating that is about 10 μm or less in thickness. A thin coating is particularly advantageous for medical articles such as catheters, wherein it is desirable to not significantly reduce the usable space within the lumen of the catheter.

Because of its extremely low friction coefficient, the halogenated nano- or microparticulate coating, particularly PTFE nano- or microparticulate coatings, can significantly reduce frictional forces associated with moving parts. In some aspects of the invention, the inventive coating is used in a medical device system comprising two or more medical articles. One (a first) of the medical articles includes the low friction microparticle-containing coating, and another (the second) medical article is moved in contact with the low friction microparticle-containing coating. The system can be used in a method involving the insertion of the medical article into a portion of the body.

One exemplary combination includes a medical system comprising a catheter and a stent. The catheter can include a halogenated nano- or microparticulate coating on its inner (diameter) surface. The stent can be loaded into a portion of the catheter and in contact with the coating.

In a related aspect, the invention provides methods for reducing the push force associated with the deployment of an implantable medical device. The method comprises a step of providing a catheter having an inner diameter coating. The inner diameter coating includes a halogenated polymeric nano- or microparticle coated layer which contacts a medical device. The method also comprises a step of providing an implantable device in contact within the inner diameter coating, and another step of moving the implantable device in contact within the inner diameter coating. The halogenated polymeric nano- or microparticle coating reduces the frictional forces associated with movement of the device.

### BRIEF DESCRIPTION OF THE DRAWING

FIG. 1 is a scanning electron microscope (SEM) image (5000×) of a coating with an outer layer of tightly packed PTFE particles (200-300 nm average diameter) formed on a polystyrene substrate.

### DETAILED DESCRIPTION

The embodiments of the present invention described herein are not intended to be exhaustive or to limit the invention to the precise forms disclosed in the following detailed description. Rather, the embodiments are chosen and described so that others skilled in the art can appreciate and understand the principles and practices of the present invention.

All publications and patents mentioned herein are hereby incorporated by reference. The publications and patents disclosed herein are provided solely for their disclosure. Nothing herein is to be construed as an admission that the inventors are not entitled to antedate any publication and/or patent, including any publication and/or patent cited herein.

Generally, the coatings of the invention include halogenated nano- or microparticles that are stably associated with a surface of the article to form a coating. The coatings include halogenated nano- or microparticles coupled to a coupling component that facilitates the formation of a coating with desirable properties on a surface of an article.

The present invention is also directed to methods for preparing low friction nano- or microparticulate coatings on the surface of articles. The methods of the present invention can be performed to provide a low frictional surface to any suitable article. However, in order to describe the invention, the low-friction coatings are more specifically discussed in the context of coatings for the surfaces of insertable medical devices. The nano- or microparticulate coatings of the present invention can provide distinct advantages that are particular desirable in the use and preparation of insertable medical devices. These desirable properties include biocompatibility, compliance, durability, and flexibility.

Insertable medical articles broadly refer to those that are placed within the body temporarily, or for longer periods of time, such as to exert a prolonged therapeutic effect in the body. Those that are placed in the body for longer periods of time can be considered “implantable”. Insertable medical articles typically come into contact with body fluids and/or tissue during the insertion process. The insertable medical article can be one that is implanted temporarily or permanently into a mammal for the prophylaxis or treatment of a medical condition. For example the insertable medical article can be introduced subcutaneously, percutaneously, or surgically to rest within an organ, tissue, or lumen within a mammal.

Insertable medical articles also include those that are partially inserted into the body to facilitate the insertion or implantation of another medical article. Such an article can be referred to as a delivery device or instrument, and can be used in an implantable device delivery system. An exemplary delivery device/implantable device system is a catheter/stent combination. Other components such as guidewires, pushwires, and sheaths can be included in the delivery system. The low friction nano- or microparticulate coatings of the invention can be formed on one or more of the devices of the delivery system and can be used to reduce the frictional forces that are associated with the movement of members of the implantable device delivery system.

The following is an exemplary list of devices that the low friction nano- or microparticulate coating can be formed on, or which are devices that can be associated with a delivery device that can have a low friction nano- or microparticulate coating. One of skill in the art can use the teachings herein to form the inventive coatings on other devices, if desired. Exemplary medical articles include vascular implants and grafts, grafts, surgical devices; synthetic prostheses; vascular prosthesis including endoprosthesis, stent-graft, and endovascular-stent combinations; small diameter grafts, abdominal aortic aneurysm grafts; wound dressings and wound management devices; hemostatic barriers; mesh and hernia plugs; patches, including uterine bleeding patches, atrial septic defect (ASD) patches, patent foramen ovale (PFO) patches, ventricular septal defect (VSD) patches, and other generic cardiac patches; ASD, PFO, and VSD closures; percutaneous closure devices, mitral valve repair devices; left atrial appendage filters; valve annuloplasty devices, catheters; central venous access catheters, vascular access catheters, abscess drainage catheters, drug infusion catheters, parenteral feeding catheters, intravenous catheters (e.g., treated with antithrombotic agents), stroke therapy catheters, blood pressure and stent graft catheters; anastomosis devices and anastomotic closures; aneurysm exclusion devices; biosensors including glucose sensors; cardiac sensors; birth control devices; breast implants; infection control devices; membranes; tissue scaffolds; tissue-related materials; shunts including cerebral spinal fluid (CSF) shunts, glaucoma drain shunts; dental devices and dental implants; ear devices such as ear drainage tubes, tympanostomy vent tubes; ophthalmic devices; cuffs and cuff portions of devices including drainage tube cuffs, implanted drug infusion tube cuffs, catheter cuff, sewing cuff; spinal and neurological devices; nerve regeneration conduits; neurological catheters; neuropatches; orthopedic devices such as orthopedic joint implants, bone repair/augmentation devices, cartilage repair devices; urological devices and urethral devices such as urological implants, bladder devices, renal devices and hemodialysis devices, colostomy bag attachment devices; and biliary drainage products.

In some specific aspects the low friction nano- or microparticulate coating is formed on a device that is used in a process involving the insertion of a medical article into a portion or portions of the cardiovascular system, such as an artery, vein, ventricle, or atria of the heart.

In other specific aspects the low friction nano- or microparticulate coating is formed on a device that is used in a process involving the insertion of a medical article in the urogenital system, such as the urethra or ureter. For example, the nano- or microparticulate coating can be formed on the inner diameter of a guide catheter for the insertion of a ureteral or urethral stent.

In other specific aspects the low friction nano- or microparticulate coating is formed on a device that is used in a process involving the insertion of an occlusion device into a portion of the body. For example, the occlusion device can be selected from vascular occlusion coils, wires, braids, strings, and the like; some vascular occlusion devices have a helically wound configuration. Commonly used occlusion devices are those that are inserted into aneurysms. Some specific vascular occlusion devices include detachable embolization coils, such as those described by Gugliemli in U.S. Pat. Nos. 5,122,136 and 5,354,295.

The low friction nano- or microparticulate coatings of the invention can be formed on a wide variety of materials (i.e., materials that have been used to fabricate the medical article or device). The devices described herein can be fabricated from one or more of these materials, or other materials known in the art and used to fabricate medical articles.

In order to define the material that is used to fabricate a coated medical article, the materials that form the structure of the article are referred to herein as “article materials” or “device materials” whereas the materials used to form the nano- or microparticulate coatings are herein referred to as “coating materials.” In many cases, the medical article is formed from one or more biomaterial(s), as the coated article is typically placed in contact with biological fluids or tissues following implantation in the body.

The low friction nano- or microparticulate coating of the present invention can be formed on surface of an insertable medical article fabricated from a single biomaterial or a combination of biomaterials. Commonly used biomaterials include plastic and/or metal materials that elicit little or no adverse biological response when placed within the body.

In some cases, the low friction nano- or microparticulate coating is formed on an insertable medical article that is fabricated from one or more plastic materials. Exemplary plastic materials include polyvinylchloride (PVC), polytetrafluoroethylene (PTFE), polyethersulfone (PES), polysulfone (PS), polypropylene (PP), polyethylene (PE), polyurethane (PU), polyetherimide (PEI), polycarbonate (PC), and polyetheretherketone (PEEK).

The low friction nano- or microparticulate coating can also be formed on medical articles fabricated partially or solely from metals. Metals that are commonly used in medical articles include platinum, gold, or tungsten, as well as other metals such as rhenium, palladium, rhodium, ruthenium, titanium, nickel, and alloys of these metals, such as stainless steel, titanium/nickel, nitinol alloys, and platinum/iridium alloys. Additional coating components, such as polymeric materials that adhere well to a metal surface can be used to facilitate formation of the low friction nano- or microparticulate coating.

Although many devices or articles are constructed from substantially all metal materials, such as alloys, some may be constructed from both non-metal and metal materials, where at least a portion of the surface of the device is metal. The metal surface may be a thin surface layer. Such surfaces can be formed by any method including sputter coating metal onto all or portions of the surface of the device.

Other surfaces that can be coated using the methods of the present invention include those that include human tissue such as bone, cartilage, skin and teeth; or other organic materials such as wood, cellulose, compressed carbon, and rubber. Other contemplated biomaterials include ceramics including, but not limited to, silicon nitride, silicon carbide, zirconia, and alumina, as well as glass, silica, and sapphire. Combinations of ceramics and metals can also be coated.

Generally, the coating of the invention includes a first coated layer including the coupling component, and a second coated layer that includes the halogenated polymeric nano- or microparticles that are coupled to the coupling component. However, the coating may also optionally include coated layers other than the layer that includes the coupling component and the layer of halogenated nano- or microparticles.

As used herein, the term “layer” or “coated layer” will refer to a layer of one or more coated materials of sufficient dimensions (for example, thickness and area) for its intended use over the entire, or less than the entire, portion of an article surface. A layer of nano- or microparticles can also be a coated layer. Therefore, a “coating” as described herein can include one or more “coated layers,” each coated layer including one or more coating components. It is also understood that during the coating process, in some cases, materials from one coated layer may migrate into adjacent coated layers, depending on the components of a particular coating composition, including the solvent or solution, and dissolved or suspended coating compounds. Therefore, to a certain extent, a coated layer may contain components from an adjacent coated layer.

One or more additional optional coated layers can be included in the coating on the article. Generally, if one or more additional optional coated layers are present in the coating, the additional layer(s) are located between the microparticle layer and the surface of the device, and more typically between the first coated layer including the coupling component and the surface of the device. Therefore, when referring to the step of disposing the coupling component on a surface, the surface may be that of the device itself, or the surface of the device with one or more optional coated layers.

Other optional coated layers, such as primer layers, that can include a non-polymeric silane compound. Exemplary silane precoatings which can be used as a primer layer in the coating of the present invention are described in U.S. Pat. No. 6,706,408.

To exemplify the coating process and benefits that the nano- or microparticulate coatings of the present invention provide to medical devices, low friction coatings on the surface of a self-expanding stent delivery catheter are discussed. Stents are typically deployed via a catheter delivery system to a target site to maintain vascular patency. Self-expanding stent delivery catheters are commonly used for the delivery of a self-expanding stent to a target location via the vasculature. These catheters are commonly used in percutaneous transluminal coronary angioplasty (PTCA) procedures. PTCA can increase blood flow through the coronary artery and is often be used as an alternative to coronary by-pass surgery.

The catheter (i.e., the catheter tubing) can be fabricated from any suitable biomaterial. Generally, it is desired to use materials that provide a suitable surface for deposition of the materials used for forming the low friction nano- or microparticulate coating. In some aspects the catheter includes a thermoplastic material such as PEBAX, polyurethane, polyethylene, polyamide, or combinations thereof The catheter can be prepared using thermoplastic materials in combination with metal wires coils, such as nitinol wire coils, and the like.

The properties of the catheter can also differ along its length. For example, the thickness of the catheter tubing can vary from its proximal end to its distal end. A catheter that is thicker at its proximal end can also be stiffer; the relative stiffness of the catheter can decrease towards the distal end. This can allow the catheter to be easier to manipulate and control as the distal end is advanced towards the target site. The low friction nano- or microparticulate coating can be present at any one or more locations on the catheter.

As exemplified by a self-expanding stent delivery catheter, the low friction microparticulate coating of the invention can improve function of the device. A coating on the internal diameter of the catheter can facilitate movement of the stent within and out of inner diameter, reducing the frictional forces during the insertion process.

Prior to disposing a coating composition on the surface of the article, such as an inner diameter of a catheter, the article can be cleaned using any suitable technique.

The first step includes disposing a coupling component on a surface of the article, such as an inner wall of a catheter. The coupling component has properties suitable for formation of a compliant coating and coupling of the halogenated nano- or microparticles. In many aspects, the coupling component is a polymeric material having groups that are reactive with the pendent groups on the nano- or microparticle.

In some desired modes of practice, a step in the coating process involves disposing the coupling component, which is a polymer that is synthetic and that has a first reactive group, wherein the polymer is also soluble in a polar liquid. The polymer is herein referred to as the “first polymer” for convenience of discussion. The polymer is “film forming” and has the properties of being able to be disposed on the surface of an article and form a coated layer. The first polymer can be a homopolymer or a copolymer having a first reactive group. The first reactive group of the polymer can react with the second reactive group of the halogenated nano- or microparticle. In some aspects the first reactive groups can be selected from carboxylate-reactive, amine-reactive, and sulfhydryl-reactive groups. Preferred first reactive group are carboxylate-reactive and can be selected from carbodiimide (—N═C═N—) or carbodiimide-containing groups.

In some aspects the first reactive groups of the water-soluble polymer of the first coated layer are carbodiimide groups. A water-soluble poly(carbodiimide) (pCDI) refers to a polymer that includes carbodiimide groups (—N═C═N—) that can be dissolved in water. Such poly(carbodiimides) can be formed by the polymerization of monomers having isocyanate groups (O═C═N—), such as m-tetramethylxylylene diisocyanate, wherein the poly(carbodiimide) is further modified with a hydrophilic portion that provide the polymer with water soluble properties. The hydrophilic portion can be cationic and include, for example, a quaternary amine group, anionic and include, for example, a sulfonate group, or nonionic and include, for example, polyether or polyester portions.

Water-soluble pCDIs can be coated on a wide variety of substrates. These include substrates those formed from a thermoplastic material, such as polyvinyl chloride (PVC) or latex. The coating can be performed using a coating composition that allows direct application and adherence of the pCDI to the thermoplastic substrate surface. Other substrates include those formed from metal or metal alloys. Metal or metal alloy substrates may include a silane primer to facilitate formation of the pCDI layer.

Suitable poly(carbodiimide) polymers for the first coated layer are available under the trade name Carbodilite™ commercially available from Nisshinbo Chemical and also described in U.S. Pat. No. 5,688,875.

In some aspects, the polymer of the first coated layer can include comonomers such as vinyl monomers and/or monomers that include aliphatic or non-polar groups.

A first coating composition can be prepared that includes the first polymer with a first reactive group, such as poly(carbodiimide), in an amount sufficient for the formation of a coated layer on the surface of the article. The coating composition including the first polymer preferably has a viscosity that is suitable for the type of coating process performed. In order to prepare a coating composition, the first polymer and any other optional component, can be dissolved or suspended in a suitable polar liquid. Exemplary polar liquids include alcohol or water. In preferred aspects, the viscosity of the coating composition is in the range of about 5 to 200 cP (at about 25° C.).

In some modes of practice the first polymer is dissolved or suspended at a concentration in the range of about 5% to about 20% (about 50-200 mg/mL); and in more specific modes of practice about 5% to about 15%. In some aspects, if more than one polymer is present in the first coating composition, the combined amount of polymeric materials can be in the ranges as described. In one exemplary preparation, first coating composition includes poly(carbodiimide) at a concentration of about 10%.

In some aspects of the invention, a water soluble poly(carbodiimide) is included in the first coating composition. According to the invention, the water soluble poly(carbodiimide) is soluble in different polar liquids, including aqueous liquids (e.g., water and buffered water solutions), alcohol (such as isopropanol or ethanol), tetrahydrofuran (THF), toluene, and methyl ethyl ketone (MEK). One or more liquids can be chosen to provide a coating having a first coated layer with desired properties, such as a desired thickness. For example, a water soluble poly(carbodiimide) can be dissolved in a composition containing water or an alcohol to provide a thinner coating, or can be dissolved in THF, toluene, or MEK to provide a thicker coating. When the first coating composition is disposed on a substrate that includes a material such as PVC or latex, the THF, toluene, or MEK-based compositions can swell the substrate material. The swelling can cause the first polymer to become at least partially incorporated into the substrate material and can therefore improve the durability of the coating.

In some modes of practice, the first coating composition includes a poly(carbodiimide) and an amount of alcohol, such as IPA, of about 30% or greater, and in more specific modes in the range of about 30% to about 70%. Alcohol-based compositions are desired as they are able to provide good wetting to substrates and also evaporate after the composition has been disposed on the surface.

The coating process can be carried out at a temperature suitable to provide a coating to the surface, or a portion of the surface, of the article or device. Preferably, the coating process is carried out at a temperature in the range of 10° C. to 50° C., and more preferably at a temperature in the range of 15° C. to 25° C. However, the actual coating temperature can be chosen based on aspects of the first coating composition. The coating temperature can also be chosen based, in some modes of practice, on the liquid used to dissolve or suspend the polymeric material, the polymeric material, and also the method used to dispose the first coating composition on the surface of the article or device.

The first coating composition can be applied to the surface of a device using any suitable technique. For example, the first coating composition can be dipped, sprayed, sponged, or brushed on a device to form a layer, and then dried.

A straightforward method for applying the coating composition is by dip-coating. A typical dip-coating procedure involves immersing the article to be coated in the first coating composition, dwelling the object in the composition for a period of time (a standard time is generally less than about 30 seconds, and can even be less that 10 seconds in many cases), and then removing the article from the composition. After the article has been dip-coated in the coating solution, it is removed and dried. Drying can be carried out using any suitable method, including air-drying the dip coated article. Times up to 30 minutes can be sufficient to dry the coated article although shorter times may be also sufficient.

Optionally, the process can be repeated to provide a coating having multiple coated layers (multiple layers formed from the first coating composition). The suitability of the coating composition for use with a particular medical article, and in turn, the suitability of the application technique, can be evaluated by those skilled in the art, given the present description.

The methods of the invention allow for the formation of a relatively thin, but very durable coating. In an exemplary preparation, the first coated layer has a thickness in the range of up to about 5 μm or about 7 μm (coating conditions can be altered or repeated to increase the thickness, such as up to about 10 μm) in a dried state. In addition to varying the liquid in the composition, the thickness of the coating can also be affected by changing the concentration of the polymer in solution. That is, increasing the concentration of the polymer can provide a thicker first coated layer, while decreasing the concentration of the polymer can provide a thinner first coated layer. The first coated layer is also compliant and conformal, meaning that it shapes well to the article to which it has been coated on, and that it can form to the changes in the shape of the device without introducing any substantial physical deformities.

Optionally, a cross-linking agent is included in the first coating composition. The crosslinking agent can include two or more latent reactive groups. The latent reactive groups are activated when exposed to an appropriate activating source and can form bonds between the materials within the coating and/or the device surface.

Use of a crosslinking agent including latent reactive groups can improve the coating in various ways. For example, the crosslinking agent can improve the durability of the coating by creating additional coupling between the coating components and/or the coating components and the surface of the coated article. Activation of the latent reactive groups of the crosslinking agent is also thought to drive reaction between the first reactive groups of the coupling component and the second reactive groups present on the microparticle. This in turn improves coupling between the coupling components and the nano- or microparticles and improves durability of the coating. In addition, use of a crosslinker with latent reactive groups is thought to promote the formation of a coating having an increased density of polymeric material.

The use of a crosslinking agent with latent photoreactive groups can represent an improvement over conventional crosslinking agents which may be reactive with specific chemical groups, and which may not react with article materials.

If included in the first coating composition, the crosslinking agent can be included at a concentration that can improve the properties of the coating. For example, the cross-linking agent can be added in an amount to improve the durability, wetting properties, or resistance to reduction in the wettability as caused by sterilization processes. Exemplary amounts of the cross-linking compound present in the coating composition range from about 0.1% to about 3%, or about 0.5% to about 2.5% weight/volume (w/v). An exemplary amount of cross-linking agent added to the present coating composition is about 1% weight/volume (w/v).

The photoactivatable cross-linking agent can be ionic, and can have good solubility in an aqueous composition. Thus, in some embodiments, at least one ionic photoactivatable cross-linking agent is used to form the coating.

Any suitable ionic photoactivatable cross-linking agent can be used. In some embodiments, the ionic photoactivatable cross-linking agent is a compound of formula I:

X₁—Y—X₂

where Y is a radical containing at least one acidic group, basic group, or a salt of an acidic group or basic group. X₁ and X₂ is each independently a radical containing a latent photoreactive group.

As an example, the photoreactive group can be an aryl ketone, such as acetophenone, benzophenone, anthraquinone, anthrone, quinone, and anthrone-like heterocycles. Spacers can also be part of X₁ or X₂ along with the latent photoreactive group. In some embodiments, the latent photoreactive group includes an aryl ketone or a quinone.

The radical Y in formula I provides the desired water solubility for the ionic photoactivatable cross-linking agent. The water solubility (at room temperature and optimal pH) is at least about 0.05 mg/mL. In some embodiments, the solubility is about 0.1 to about 10 mg/mL or about 1 to about 5 mg/mL.

In some embodiments of formula I, Y is a radical containing at least one acidic group or salt thereof. Such a photoactivatable cross-linking agent can be anionic depending upon the pH of the coating composition. Suitable acidic groups include, for example, sulfonic acids, carboxylic acids, phosphonic acids, and the like. Suitable salts of such groups include, for example, sulfonate, carboxylate, and phosphate salts. In some embodiments, the ionic cross-linking agent includes a sulfonic acid or sulfonate group. Suitable counter ions include alkali, alkaline earths metals, ammonium, protonated amines, and the like.

For example, a compound of formula I can have a radical Y that contains a sulfonic acid or sulfonate group; X₁ and X₂ can contain photoreactive groups such as aryl ketones and quinones. Such compounds include 4,5-bis(4-benzoylphenylmethyleneoxy)benzene-1,3-disulfonic acid or salt; 2,5-bis(4-benzoylphenylmethyleneoxy)benzene-1,4-disulfonic acid or salt; 2,5-bis(4-benzoylmethyleneoxy)benzene-1-sulfonic acid or salt; N,N-bis[2-(4-benzoylbenzyloxy)ethyl]-2-aminoethanesulfonic acid or salt, and the like. See U.S. Pat. No. 6,278,018. The counter ion of the salt can be, for example, ammonium or an alkali metal such as sodium, potassium, or lithium.

In other embodiments of formula I, Y can be a radical that contains a basic group or a salt thereof. Such Y radicals can include, for example, an ammonium, a phosphonium, or a sulfonium group. The group can be neutral or positively charged, depending upon the pH of the coating composition. In some embodiments, the radical Y includes an ammonium group. Suitable counter ions include, for example, carboxylates, halides, sulfate, and phosphate.

For example, compounds of formula I can have a Y radical that contains an ammonium group; X₁ and X₂ can contain photoreactive groups that include aryl ketones. Such photoactivatable cross-linking agents include ethylenebis(4-benzoylbenzyldimethylammonium) salt; hexamethylenebis (4-benzoylbenzyldimethylammonium) salt; 1,4-bis(4-benzoylbenzyl)-1,4-dimethylpiperazinediium) salt, bis(4-benzoylbenzyl)hexamethylenetetraminediium salt, bis[2-(4-benzoylbenzyldimethylammonio)ethyl]-4-benzoylbenzylmethylammonium salt; 4,4-bis(4-benzoylbenzyl)morpholinium salt; ethylenebis[(2-(4-benzoylbenzyldimethylammonio)ethyl)-4-benzoylbenzylmethylammonium] salt; and 1,1,4,4-tetrakis(4-benzoylbenzyl)piperzinediium salt. See U.S. Pat. No. 5,714,360. The counter ion is typically a carboxylate ion or a halide. On one embodiment, the halide is bromide.

In some aspects a non-ionic photoactivatable cross-linking agent can be used. In one embodiment, the non-ionic photoactivatable cross-linking agent has the formula XR₁R₂R₃R₄, where X is a chemical backbone, and R₁, R₂, R₃, and R₄ are radicals that include a latent photoreactive group. Exemplary non-ionic cross-linking agents are described, for example, in U.S. Pat. Nos. 5,414,075 and 5,637,460 (Swan et al., “Restrained Multifunctional Reagent for Surface Modification”). Chemically, the first and second photoreactive groups, and respective spacers, can be the same or different.

Some suitable cross-linking agents are those formed by a mixture of the chemical backbone molecule (such as pentaerythritol) and an excess of a derivative of the photoreactive group (such as 4-bromomethylbenzophenone). An exemplary product is the tetrakis (4-benzoylbenzyl ether) of pentaerythritol (tetrakis(4-benzoylphenylmethoxy-methyl)methane). See U.S. Pat. No. 5,414,075 (columns 7 and 8, lines 1-25 (Formula III) and U.S. Pat. No. 5,637,460.

A single photoactivatable cross-linking agent or any combination of photoactivatable cross-linking agents can be used in forming the coating. In some embodiments, at least one nonionic cross-linking agent such as tetrakis(4-benzoylbenzyl ether) of pentaerythritol can be used with at least one ionic cross-linking agent. For example, at least one non-ionic photoactivatable cross-linking agent can be used with at least one cationic photoactivatable cross-linking agent such as an ethylenebis(4-benzoylbenzyldimethylammonium) salt or at least one anionic photoactivatable cross-linking agent such as 4,5-bis(4-benzoyl-phenylmethyleneoxy)benzene-1,3-disulfonic acid or salt. In another example, at least one nonionic cross-linking agent can be used with at least one cationic cross-linking agent and at least one anionic cross-linking agent. In yet another example, a least one cationic cross-linking agent can be used with at least one anionic cross-linking agent but without a non-ionic cross-linking agent.

The choice of a crosslinking agent may depend on the ingredients in the first or second coating composition. For example, a first coating composition that includes a poly(carbodiimide) in an aqueous liquid preferably includes an anionic crosslinking agent. However, a first coating composition that includes a poly(carbodiimide) in a liquid such as an alcohol or liquid such as THF, MEK, or toluene, preferably includes a nonionic crosslinking agent.

If a cross-linking agent having latent reactive groups is included in the first coating composition, in some cases a step of irradiating may be performed to activate the latent reactive group. For example, the coating can be treated with UV irradiation following the step of disposing a first coating composition that includes a poly(carbodiimide) and an ionic photoactivatable cross-linking agent. The step of activating can be performed before and/or after the first coated layer dries. However, the step of activating may be performed two or more times during the coating process.

Generally, the step of irradiating can be performed by subjecting the photoreactive groups to actinic radiation in an amount that promotes activation of the photoreactive group and coupling to a target moiety. In preferred aspects, the step of irradiating is performed after the second coating composition is disposed.

Actinic radiation can be provided by any suitable light source that promotes activation of the photoreactive groups. Preferred light sources (such as those available from Dymax Corp.) provide UV irradiation in the range of 190 nm to 360 nm. A suitable dose of radiation is in the range of from about 0.5 mW/cm² to about 2.0 mW/cm².

In some aspects, it may be desirable to use filters in connection with the step of activating the photoreactive groups. The use of filters can be beneficial from the standpoint that they can selectively minimize the amount of radiation of a particular wavelength or wavelengths that are provided to the coating during the activation process. This can be beneficial if one or more components of the coating are sensitive to radiation of a particular wavelength(s), and that may degrade or decompose upon exposure.

After the first polymer is disposed, the halogenated nano- or microparticles having a second reactive group are disposed on the first layer. A portion of the second reactive groups becomes covalently coupled to the first reactive groups forming a reacted pair, coupling the microparticle to the first component. The second coated layer can be formed by preparing a coating composition that includes the halogenated nano- or microparticles having second reactive groups.

The halogenated nano- or microparticles of the invention can have any three-dimensional structure. Typically, the nano- or microparticles will have rounded surfaces; i.e., edges as would otherwise be observed on cubed structures, are generally not present. In many cases the nano- or microparticles can be spherical, but many other structures with rounded surfaces are possible, such as spheroid or ellipsoid structures, including oblong spheroid structures, etc.

The halogenated nano- or microparticles are of a size measured in nanometers or micrometers in diameter (the average cross-sectional dimension of the particle). In some selected modes of practice, the coating is formed using halogenated nano- or microparticles having a size of about 50 nm or greater in diameter. The nano- or microparticulate layer can be formed having a thickness sufficient to withstand abrasion due to movement against another surface.

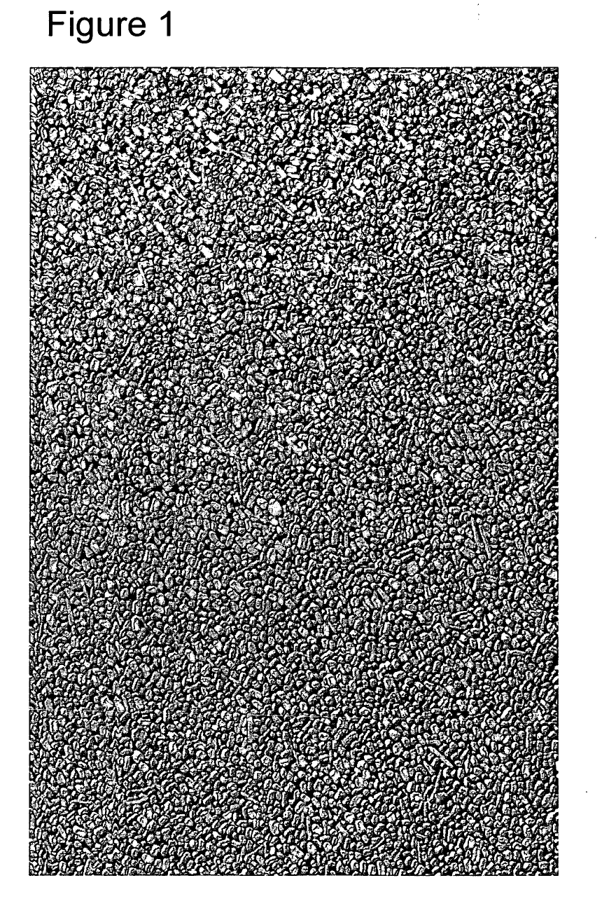

In another selected mode of practice, the coating is formed using halogenated nanoparticles having a size in the range of about 50 nm to about 500 nm, and more specifically in the range of about 100 nm to about 300 nm. A particulate coating having a size within this range can provide particularly desirable surface properties. Due to these relatively smaller sizes, the nano- or microparticles can become densely packed in the nano- or microparticulate layer. The small size of the nano- or microparticles affords a smooth surface (see FIG. 1). Furthermore, a greater percentage of the nano- or microparticulate surface area is available for contact with the first coated layer that includes the coupling component (such as the water soluble polymer with first reactive groups). This increase of microparticle surface area provides that a greater percentage of second reactive groups on the microparticle are available for coupling with the first reactive groups of the first coated layer and therefore increases the stability of the nano- or microparticles, and overall durability of the coating. In addition, as compared to larger particles, the relatively smaller sized nano- or microparticles are less likely to become dislodged from the surface due to sheer forces encountered upon movement of a device over the nano- or microparticulate surface.

The halogenated microparticle with pendent reactive groups can be formed according to methods described in the prior art (see, for example, U.S. Pat. No. 7,041,728). For example, the halogenated microparticle with pendent reactive groups can be formed using an aqueous emulsion polymerization of a halogenated monomer, such as tetrafluoroethylene (TFE). In the final stages of this emulsion polymerization, free radicals that can introduce ionic end groups or precursors thereof are introduced into the polymerization reaction sufficient to cause the rate of polymerization to increase by at least 20%.

The resulting halogenated nanoparticle or microparticle includes the ionic groups (herein referred to as second reactive groups), such as carboxylate groups, that are present on the surface of the microparticle, and which can be reacted with the first reactive groups of the coupling component in the coating process of the present invention. The amount of pendent first reactive groups on the surface of the halogenated nanoparticles or microparticles are sufficient to form a low friction coating on the surface of an article, such as a catheter.

A desired nanoparticle or microparticle is formed predominantly from a perfluorinated polymer. An exemplary perfluorinated polymer is PTFE. The microparticle can also include, or be formed from, other halogenated monomers. Other perhalogenated monomers include chlorotrifluoroethylene (CTFE), fluorinated ethylene (FE), and hexafluoropropylene. Examples of partially fluorinated monomers include vinylidene fluoride (VDF) and perfluoro alkyl or alkoxy vinyl ether monomers.

The co-monomer used to provide pendent first reactive groups on the surface of the nanoparticle or microparticle includes a perfluorinated co-monomer with acid groups or salts thereof such as carboxylic acid, sulfonic acid, phosphoric or phosphonic acid and salts thereof, as well as amine and sulfhydryl groups. Exemplary co-monomers also include perfluorinated allyl or vinyl ether having one or more ionic groups or precursors thereof.

In the coating composition, the halogenated nano- or microparticles can be suspended in a suitable liquid. In one mode of practice the nano- or microparticles are suspended in polar liquid, such as water. The nano- or microparticles can be present at a concentration that allows the formation of a nano- or microparticulate layer sufficient to achieve a low-friction uniform coating. In some modes of practice, the nano- or microparticles are present in the composition at about 10 g/L or greater, and in some desired modes, in the range of about 10 g/L to about 400 g/L. At these concentrations, the number of nano- or microparticles per unit volume will depend on the size and density of the nano- or microparticles.

Such a density can allow formation of a tightly packed nano- or microparticulate layer. In such a layer, as visualized under microscopy, such as SEM, the nano- or microparticles form a coherent layer, without significant gaps in the layer. In a tightly packed nano- or microparticulate layer, the nano- or microparticles will have surfaces in contact with one another.

The nano- or microparticles will have a surface that is in contact with the layer comprising the coupling component, wherein the first and second reactive groups have been reacted. This surface of the nano- or microparticles can be referred to as the coupled surface. The microparticle will also have surfaces that are in contact with other nano- or microparticles.

Generally the nano- or microparticulate layer will have a thickness of about 2 μm or less. In exemplary preparations, the nano- or microparticulate layer will have a thickness of about 1 μm or less. The thickness of the coated layer can depend on the average microparticle size used. In some aspects, the thickness can be substantially less than 1 μm, and can be as thin as about 200 nm. In one exemplary preparation the microparticle coated layer has a thickness of about 500 μm.

The microparticle layer may have the appearance of a monolayer of nano- or microparticles. However, some nano- or microparticles may become partially or fully embedded in the material of the first coated layer. This can increase the apparent thickness of the microparticle layer depending on the analysis technique used.

For coatings having the first (coupling component) and second (nano- or microparticulate) layers, the overall coating thickness can be less than about 10 μm. In some preparations the coating has a thickness in the range of about 4 μm to about 8 μm. An exemplary coating is formed from a coupling component that is a water soluble polycarbodiimide polymer, and PTFE nano- or microparticles having an average size in the range of about 200 nm-300 nm. The PTFE nano- or microparticles are coupled to the polycarbodiimide polymer via reacted carboxylate groups pendent from the PTFE microparticle.

In a related aspect, the article can include a hydrophilic, lubricious coating in addition to the halogenated microparticle coating. Such an article will therefore have at least two different low friction surfaces, one being a “dry” low friction surface as formed by the halogenated microparticle layer, and the other being a “wet” low friction as formed by a hydrophilic polymer.

In some desired modes of practice, the coating is formed using a common coupling component having first reactive groups. In forming the nano- or microparticulate and hydrophilic coated surfaces, halogenated nano- or microparticles and a hydrophilic polymer have pendent reactive groups (such as second reactive groups) that are specifically reactive with the first reactive groups of the coupling component. Advantageously, this allows the coating having different low friction surfaces to be prepared in a very efficient and effective manner.

The process is exemplified describing the preparation of a catheter having a low friction halogenated nano- or microparticulate inner diameter coating, and a low friction hydrophilic outer diameter coating. Such a coated catheter is particularly useful as the hydrophilic coating on the outer diameter facilitates the movement of the catheter within a vessel, and the nano- or microparticulate inner diameter coating facilitates movement of a medical device such as a stent within the catheter.

To exemplify such a process, a coating composition including a water-soluble polymer with first reactive groups, such as pCDI, is prepared. The inner (diameter) and outer (diameter) surfaces of the catheter are contacted with the pCDI-based coating composition, desirably by a dip-coating method. This results in the formation of a compliant polymeric coated layer having first reactive groups.

In the following steps, the nano- or microparticulate and hydrophilic coatings are formed. The coatings can be formed at the same time or successively. In order to form the nano- or microparticulate coating, a coating composition that includes the halogenated nano- or microparticles is disposed within the inner diameter of the catheter. For example, one end of the catheter is plugged, and the halogenated microparticle composition is added to the lumen of the catheter. The entire lumen can be filled with the halogenated microparticle composition, or a portion of the catheter can be filled to provide a nano- or microparticulate coating on a portion of the inner diameter. For example, the process can be carried out to coat a distal portion of the inner diameter of the catheter, sufficient to contact a stent that is loaded in the distal end of the catheter.

Upon contact with the microparticle-containing composition within the lumen, the reactive groups present on the microparticle react with the first reactive groups of the first coated layer, thereby forming a nano- or microparticulate layer. The microparticle-containing composition can then be removed from the inner lumen of the catheter.

As another step in the method, a coating composition including the hydrophilic polymer with groups that are reactive with the first reactive groups is placed in contact with the outer (diameter) surface of the catheter. In some aspects, the hydrophilic polymer has the same reactive groups as the halogenated microparticle. In a desired mode of practice both the nano- or microparticles and the hydrophilic polymer include carboxylate groups as second reactive groups. However, different second reactive groups can be used on the microparticle and the hydrophilic polymer to achieve the same coating effect.

In some modes of practice, the coating is formed using a hydrophilic polymer having carboxylate groups as well as pendent ester groups (—COOR₁). The pendent ester groups can have different alkyl chain lengths (R₁), wherein R₁ can be a short chain alkyl group such as a C₁-C₄ alkyl group. Preferred carboxylate and ester group-containing hydrophilic copolymers can also be obtained by copolymerizing a vinyl ether, such as methyl vinyl ether, with maleic anhydride, and then reacting the copolymer with an alcohol to produce a copolymer having ether, ester, and carboxylate groups. One preferred and exemplary copolymer is a copolymer of methyl vinyl ether and maleic anhydride, wherein the copolymer is reacted with a C₂-C₄ alcohol. These copolymers can be commercially obtained under the trade name of Gantrez™ ES (for example Gantrez™ ES 225 or Gantrez™ ES 425) from International Specialty Products (Wayne, N.J.). These hydrophilic coating are also described in commonly assigned co-pending U.S. patent application Ser. No. 11/445,806, filed Jun. 2, 2006, and entitled “Hydrophilic Polymeric Coatings for Medical Articles.”

As another optional feature, the nano- or microparticle containing coatings of the invention can also include a colorant. A colorant can be provided along portions of the article surface to allow the progress of insertion of the article device into a patient to be monitored. The colorant(s) can provide a visual cue to the end user to indicate where the coating composition is located along the coated article (in other words, what portions of the device surface are in fact provided with a coating composition). The presence of a coating on a device surface is often determined by tactile means, meaning that the user can feel the portions of the device that are provided with a lubricious coating. A coating with a colorant can allow the user to visually determine the coated portions of the device, as compared to the more tactile methods. Being able to visually determine the coated portions of the device can improve the safety by reducing the handling of the device, which minimizes contamination by microorganisms. Further, when different coating compositions are provided on a device surface, discrete colorant can be provided for each coating composition, thereby providing a visual cue as to the identity and location of the different coating compositions.

The colorant can be present in any portion of the coating. For example, the colorant can be included in the composition that is used to form the first coated layer. The colorant can also be present in the hydrophilic coating, if optionally formed on the article.

Example of colorants that can be used in the preparation of coatings of the present invention include, but are not limited to, FD&C and D&C lakes, titanium dioxide, magnesium carbonate, talc, pyrogenic silica, iron oxides, channel black, insoluble dyes, natural colorants (such as riboflavin, carmine 40, curcumin, and annatto), dyes approved for ingestion by the U.S. Federal Drug Administration, or a combination of any of these. Colorants used in making coating dispersions for coating tablets, food, confectionery forms, agricultural seeds, and the like can be used in the coatings of the present invention.

The colorant can be present in one or more coated layers in an amount up to about 55% by weight of the non-liquid ingredients of the coating composition. In some exemplary modes of practice the colorant is used at about 1% wt/v in the coating composition. The composition that includes the colorant can also include a plasticizer. Exemplary plasticizers include propylene glycol, glycerol, and glycerin.

The coatings of the present invention can also be prepared including an imaging agent detectable when using imaging apparatus. The imaging agent can allow the coated article to be detected during a procedure, such as following the insertion of the coated instrument into a portion of the body. Examples of imaging agents include paramagnetic materials, vapor phase materials, radioisotopic materials, and radiopaque materials. The imaging agent can be associated with the layer including the coupling component, and/or the halogenated nano- or microparticles. In some cases, the halogenated microparticle can be mixed with a microparticle set that includes an imaging agent.

A suitable radiopacifying agent can be iodine, or a secondary compound, such as a commercially available iodine-containing radiopacifying agent.

After the coating has been formed on the surface of a device (such as a catheter, for example) the coated device can optionally be sterilized prior to use. While any type of sterilization procedure can be employed, a preferred procedure involves treatment with ethylene oxide. The coated device can be obtained and subject to a sterilization process, such as ethylene oxide sterilization, or a user can perform the steps of forming a hydrophilic coating and then also perform sterilizing the coated device.

Sterilization with ethylene oxide offers the advantage of avoiding the higher temperatures or the moisture associated with steam sterilization. Another advantage of ethylene oxide is that its residues volatilize relatively quickly from the article sterilized. Since ethylene oxide is a highly flammable material it is generally used in a mixture with a flame retardant. Commonly used flame retardant compounds include chlorofluorocarbons (CFCs) such as dichlorodifluoro-methane (also known as CFC 12), and carbon dioxide. Other components that can be present in mixture with ethylene oxide include inert nitrogen gas, which may be used to increase the pressure in the sterilization chamber.

An exemplary ethylene oxide sterilization is carried out as follows. The coated device is place in a commercially available sterilization chamber. The chamber is then heated to a temperature within the range of from about 54° C. (130° F.) to about 60° C. (140° F.). A partial vacuum is created in the chamber with the addition of water vapor to provide a relative humidity in the range of about 30 to about 80 percent. The sterilant mixture is then converted to a vapor and introduced into the sterilization chamber at a pressure in the range of about 362.0 millimeter of mercury (0° C.; 7 psi) to about 1706.6 millimeter of mercury (0° C.; 33 psi). The sterilization time can vary and is dependent upon a number of factors including temperature, pressure, humidity level, the specific sterilant mixture employed, and the coated device. Following exposure the ethylene oxide is evacuated from the chamber, for example, by flushing with air, nitrogen, steam or carbon dioxide.

Non-biodegradable or biodegradable stents can be used in conjunction with the stent deployment catheter having a low friction nano- or microparticulate coating as described herein. The nano- or microparticulate coating of the present invention is seen as providing a benefit to stent deployment regardless of the material that the stent is fabricated from. The self-expanding stents may be made of shape memory materials such as nitinol or constructed of regular metals but of a design that allows for self-expansion upon release from the distal end of the catheter. Examples of self-expanding stents made of shape memory materials are known in the art and can be found in, for example, U.S. Pat. Nos. 5,395,390 and 5,540,712. Examples of biodegradable stents made of biodegradable polymeric material are known in the art and can be found in, for example, U.S. Pat. No. 6,368,346. Stents can also be provided with a drug eluting and/or biocompatible coating as described in for example U.S. Application No. 2005/0244453, U.S. Pat. No. 6,214,901 (Chudzik et al.), and U.S. Pub. No. 2002/0188037 A1 (Chudzik et al.). If the stent includes a drug eluting and/or biocompatible coating, deployment from a catheter including a low-friction coating of the present invention is thought to maintain the integrity of the coating.

A self-expanding stent can be loaded in the lumen of the distal end of the delivery catheter. The surface of the lumen (i.e., the inner diameter) of the distal end is provided with a low friction nano- or microparticulate coating of the present invention. The stent is in contracted state when in the distal end of the catheter, and exerts outward pressure on the inner walls of the catheter. The ablumenal surface of the stent is in contact with the nano- or microparticulate coating when the stent is loaded into the catheter.

In a stent deployment procedure, the catheter having an inner diameter-coated distal end that includes the loaded stent is percutaneously introduced into the cardiovascular system. The distal end of the catheter is advanced to the target site. For example, in a standard PTCA procedure, the guiding catheter is advanced through the aorta until the distal end is in the ostium of the desired coronary artery. Once the distal end is at the target site, the stent is deployed from the distal end. The low friction nano- or microparticulate coating facilitates stent deployment.

A self-expanding stent can be deployed in one of a number of ways. Generally, the distal end of the catheter is moved in relation to the stent, or vice versa. For example, a deployment wire can be advanced through the catheter and placed in contact with the stent. In some modes of practice, the deployment wire is advanced distally to push the stent out of the distal end of the catheter. In other modes of practice the deployment wire maintains the placement of the stent while the catheter is retracted, thereby releasing the stent at the target site.

In other cases the low friction nano- or microparticulate coating is used in connection with a catheter for the delivery of balloon-expanded stents. The low friction nano- or microparticulate coating can also be present on the inner diameter of the distal end of the catheter to facilitate deployment of the stent from the distal end.

The low friction nano- or microparticulate coating can facilitate deployment of the stent by reducing the frictional forces associated with movement of the inner diameter of the catheter over the ablumenal surface of the stent. The coatings of the present invention can significantly reduce the push force associated with the deployment of the stent. For example, the push force can be reduced by about 50% or greater. For example, for uncoated stent delivery catheters, the push force associated with stent deployment may be about 9 newtons (N) or 10N. The low friction nano- or microparticulate coatings of the present invention are able to reduce the push force associated with stent deployment to about 5N or less.

The low friction nano- or microparticulate coating of the invention can also be formed on the inner diameters of endoscopic sheaths. Endoscopic sheaths can be used in various medical procedures, including those involving the urogenital tract, the gastrointestinal tract, and the vasculature. The coatings of the invention can reduce the frictional forces associated with the movement of an endoscope through the endoscopic sheath.

The inventive coatings can also be used to reduce frictional forces on apparatus that include, generally, a piston-cylinder combination. Examples of apparatus that include a piston-cylinder combination include engines, syringes for medical and non-medical uses, hydraulic apparatus, pneumatic apparatus, and weaponry.

For example, the coating can be formed on the surface of a syringe body/syringe plunger combination. In a manner similar to that of coating the inner diameter of a catheter, the inner diameter of the syringe body, the outer diameter of the syringe plunger, or both, can be provided with a low friction nano- or microparticulate coating of the present invention. In many cases, the syringe body is formed of a thermoplastic material and the coating of the invention is formed on the thermoplastic surface.

The coated inner diameter of the syringe body can reduce the frictional forces when the syringe plunger is moved in relation to the syringe body. This can provide a smoother and more controlled movement of the plunger body. In turn, this can improve delivery of a composition from the syringe by allowing an amount of a composition delivered from the syringe to be controlled with greater accuracy.

The coated syringe body/syringe plunger combination can be used in medical as well as non-medical applications. For applications involving delivery of the composition into a subject, the coating of the present invention also provides improved biocompatibility and is therefore well suited for these processes. In other words, the coating will not adversely affect the composition that is delivered to the subject.

The coated syringe body/syringe plunger combination can be used in non-medical applications such as in machines that involve the dispensing or spraying of a liquid composition. Some of these machines may be able to perform high throughput synthesis or analysis. In some aspects, the coated syringe body/syringe plunger combination can be used in a chemical synthesis or chemical analysis apparatus. It would be desirable to provide chemical synthesis or chemical analysis apparatus with a coating of the present invention, as these apparatus typically involve the repetitive dispensing or spraying of very small quantities of liquids. Examples of chemical synthesis apparatus include oligonucleotide synthesizers, peptide synthesizers, and polymerase chain reaction (PCR) machines.

Examples of chemical analysis apparatus include spectrometers such as matrix assisted laser desorption/ionization-time of flight (MALDI-TOF) mass spectrometers and electrospray mass spectrometers, polynucleotide sequencers, peptide sequencers, high-pressure liquid chromatography (HPLC) machines, capillary electrophoresis, surface plasmon resonance (SPR) machines, flow cytometers, cell sorters, and luminometers.

The coated syringe body/syringe plunger combination with the inventive coating can also be used in apparatus for the preparation or maintenance of microelectronics or microelectronic parts. For example, the syringe can be used to apply an encapsulant or adhesive composition to a part of a microelectronic device.

The inventive coating can also be used to coat a portion of a hydraulic or pneumatic system having an operating cylinder and piston. The coating can facilitate movement of the piston within the cylinder, and thereby may improve system function and lifetime.

The inventive coating can also be used to coat a portion of a piece of weaponry, such as the inner surface of the barrel of a gun, or a cartridge for use in association with a weapon. The coatings may facilitate operation and accuracy of the weaponry by reducing frictional forces associated with the loading and firing of the weapon.

The coatings can also be formed on articles having a telescoping function. For example, the coating can be formed on cameras or other optical equipments such as telescopes, binoculars, eyesights, and the like. The coatings can facilitate the telescoping movement by reducing frictional forces and therefore conserve battery power, if the telescoping member is mechanically driven, and can improve optical precision of the equipment.

The coatings can also be formed on articles associated with optical fibers. The coatings can be formed on a surface that is in contact with one or more optical fibers. The coated surface can be that of a sheath surrounding the fibers. The coating can also be formed on a surface of a fiber optic connector. The coating can facilitate the travel of components of a fiber optic cable in and/or out of the connector. This can improve alignment of the components and also reduce the risk of damaging the ends of the fibers.

Other contemplated uses for the coatings of the present invention are on pumps, drive train systems, recreational equipment, mechanical hosing, and wheel assemblies, such as on hubs or surfaces in contact with bearings.

### EXAMPLE 1

Ten centimeter pieces of 3 mm diameter Pellethane™ EG-60D polyurethane rods were wiped with propanol to clean. A working solution of 20% by volume polycarbodiimide (Carbodilite™ V-02-L2 from Nisshinbo Chemical, Japan) was prepared by diluting a 40% stock solution with propanol. The pieces were dipped into the working solution at a rate of 1.5 cm/second, left to soak for thirty seconds, then pulled out of the solution at a rate of 1.0 cm/second. After a fifteen minute air dry, the pieces were dipped into 30% solids PTFE particles (Dispersez™ 200W2 from PolySciences, Inc.) at 2.5 cm/second into the solution, left to soak for ten seconds, then pulled out at 1.0 cm/sec. All pieces were milky white in color after this coating. The pieces were allowed to dry at room temperature for twenty hours to cure the reaction between the carbodiimide and the grafted acrylic acid groups on the PTFE particles.

After curing, the pieces were placed in a stirring water bath to see if coating would be removed. They were given a wet vertical pinch test (details) to see if coating looked different than in uncoated Pellethane™ controls.

After forming the PTFE particulate coating on the rods polyurethane rods, the coated rods were evaluated using a Vertical Pinch Method, as described in International Application Number WO 03/055611 with the following modifications. The coated rods were inserted into the end of a rod holder, which was placed between the two jaws of a pinch tester and immersed in a cylinder of water or saline. The jaws of the pinch tester were closed as the sample was pulled in a vertical direction and opened when the coated sample was returned to the original position. A 500 g force was applied as the coated substrates were pulled up through the pinched jaws. The pull force exerted on the substrate was then measured (grams). Pull force (g) is equal to the coefficient of friction (COF) multiplied by pinch force (g). The average frictional force was determined for 5 cycles while the coated substrates traveled 10 cm at a travel rate of 1 cm/sec.

The average friction force for uncoated control was 327 grams. The coated pieces had an average force between 99 and 273 grams. Coated pieces stained intensely with toluidine blue stain.

FIG. 1 shows a scanning electron microscope (SEM) image of a coating with an outer layer of tightly packed PTFE particles (200-300 nm average diameter) formed on a polystyrene substrate.

What is claimed is:

1. A method of forming a low friction coating comprising the steps of: (a) disposing a coupling component on a surface of an article, wherein the coupling component comprises a first reactive group, and (b) disposing halogenated polymeric nanoparticles or microparticles on the coupling component, wherein the halogenated polymeric nanoparticles or microparticles comprise a second reactive group, wherein the first and second reactive groups are allowed to covalently bond, thereby coupling the halogenated polymeric nanoparticles or microparticles to the surface.

2. The method of claim 1 where, in step (a), the first reactive group is selected from hydroxyl and carboxylate-reactive groups.

3. The method of claim 2 where, in step (a), the first reactive group is a carbodiimide group.

4. The method of claim 1 where, in step (a), the coupling component comprises a polymer.

5. The method of claim 4 where, in step (a), the coupling component comprises a water-soluble polycarbodiimide.

6. The method of claim 1 where, in step (a), the coupling component is in contact with material that forms the article.

7. The method of claim 6 where, in step (a), the coupling component is at least partially impregnated in the material that forms the article.

8. The method of claim 1 where, in step (b), the halogenated polymeric nanoparticles or microparticles comprise polytetrafluoroethylene.

9. The method of claim 1 where, in step (b), the halogenated polymeric nanoparticle or microparticle has a diameter of 1 μm or less.

10. The method of claim 9 where, in step (b), the halogenated polymeric nanoparticle has a diameter in the range of 50 nm to 500 nm.

11. The method of claim 10 where, in step (b), the halogenated polymeric nanoparticle has a diameter in the range of 200 nm to 300 nm.

12. The method of claim 1 where, in step (b), the second reactive group is selected from hydroxyl and carboxylate groups.

13. The method of claim 1 where the low friction coating is formed on a device used in a medical procedure.

14. The method of claim 1 where the low friction coating is formed on an implantable or insertable medical device.

15. The method of claim 1 where the low friction coating is formed on a thermoplastic surface of the implantable or insertable medical device.

16. The method of claim 1 where the low friction coating is formed on an inner diameter surface of a catheter.

17. The method of claim 1 comprising a step of disposing a compound comprising a latent reactive group and a step of activating the latent reactive group.

18. An article comprising a low friction coating, the coating comprising:
(a) a coated layer comprising halogenated polymeric nanoparticles or microparticles, and
(b) a coupling component provided between the coated layer (a) and the article, wherein the microparticles or nanoparticles are covalently bonded to the coupling component.

19. The article of claim 18 wherein the low friction coating has a thickness of 10 μm or less in a dry state.

20. A system comprising: (a) a first article and (b) a second article, wherein either the first article, second article, or both, include a low friction coating comprising a coated layer of halogenated polymeric nanoparticles or microparticles, and wherein the coating facilitates the movement of first article in relation to the second article.

21. The system of claim 20, wherein the first article comprises the coating and the second article is fabricated of a metal or metal alloy.

22. A method comprising the system of claim 20, comprising a step of applying a force of 5N or less to cause movement of the first article in relation to the second article.
